(19) United States
(12) Patent Application Publication
(10) Pub. No.: US 2026/0215818 A1
(43) Pub. Date: Jul. 30, 2026

(54) A BONE IMPLANT SYSTEM

(71) Applicant: Randall F. Lee, Southlake, TX (US)

(72) Inventors: Randall F. Lee, Southlake, TX (US); Ian A. Lee, Carrollton, TX (US); Matthew Shomper, Xenia, OH (US); Lisa Ferrara, Hendersonville, NC (US)

(73) Assignee: Randall F. Lee, Southlake, TX (US)

(21) Appl. No.: 19/573,337

(22) Filed: Mar. 20, 2026

Related U.S. Application Data
(63) Continuation of application No. PCT/US2025/028531, filed on May 8, 2025.
(60) Provisional application No. 63/644,222, filed on May 8, 2024.
(60) Provisional application No. 63/677,230, filed on Jul. 30, 2024.

(51) Int. Cl. A61B 17/70; A61B 17/86

(52) U.S. Cl. CPC A61B 17/7032; A61B 17/8635; A61B 17/864

(57) ABSTRACT

Embodiments of a bone implant are disclosed, including a shaft having a first rotational axis; helical threads positioned about a longitudinal portion of the shaft forming a propeller region comprising a plurality of helical root valleys between crests of the helical threads, and the helical threads having distal surfaces and proximal surfaces; a plurality of pawls projecting from the distal surfaces to create a turbine-like thread-form shape; wherein the plurality of pawls are angled with respect to the distal surfaces to allow for forward rotation of the threads and to resist backward rotation of the threads; and an irregular lattice structure formed within the plurality of helical root valleys.

### CROSS-REFERENCE TO RELATED APPLICATIONS

This application is a continuation of International Application No. PCT/US2025/028531, filed May 8, 2025, entitled “A BONE IMPLANT SYSTEM,” which claims the benefit of the filing date of U.S. provisional patent application Ser. No. 63/644,222, filed on May 8, 2024, entitled “SYSTEM AND METHOD FOR A MEDICAL IMPLANT WITH AN INTEGRATED PROPULSOR,” and U.S. provisional patent application Ser. No. 63/677,230, filed on Jul. 30, 2024, entitled “SYSTEM AND METHOD FOR A MEDICAL IMPLANT WITH AN INTEGRATED PROPULSOR, the disclosures of which are incorporated herein by reference for all purposes.

### TECHNICAL FIELD

The invention relates, in general, to pedicle anchoring systems, and in particular, to pedicle anchoring systems with self-compressing propeller-shaped implants.

### BACKGROUND INFORMATION

Pedicle anchoring systems, such as pedicle screw systems, are a cornerstone of spinal fixation procedures, providing stability and support for a range of spinal pathologies, including degenerative diseases, trauma, and deformities. The success of these implants critically depends on their ability to achieve secure anchorage within the vertebrae, which is largely influenced by the quality of osseointegration (the direct structural and functional connection between the implant surface and bone tissue) and screw loosening or backout prevention.

Despite advancements in anchor design and surface modifications, challenges remain in optimizing osseointegration to enhance long-term stability and reduce the risk of screw loosening or failure. Traditional pedicle anchors often face limitations related to osseointegration failure and screw loosening, which can hinder bone-implant integration, delay healing, and increases the chance of failure.

What is needed, therefore, is a device or system that can enhance osseointegration, hinder screw loosening, improve predictability in compromised bone, and promote long-term implant stability.

### SUMMARY

In response to these and other problems, in one embodiment, there is a pedicle anchoring system comprising a propeller-shaped implant body or fixture, a head, and a rod connector.

In certain embodiments, the pedicle anchoring system comprises an anchor with a propeller having a shaft with a rotational axis; helical threads positioned about a longitudinal portion of the shaft forming a plurality of helical root valleys between crests of the helical threads, and the helical threads having distal surfaces and proximal surfaces; a plurality of pawls projecting from the distal surfaces to create a turbine-like thread-form shape; wherein the plurality of pawls are angled with respect to the distal surfaces to allow for forward rotation of the threads and to resist backward rotation of the threads; an irregular porous lattice structure may be formed within the plurality of helical root valleys; and a means for attaching the anchor to a rod or another stabilization system.

In embodiments described above, a pawl in the plurality of pawls has an angled surface facing a forward rotation direction and a concave curved surface facing away from the forward rotation direction.

In the embodiments described above, a pawl in the pluralities of pawls has a sharp edge projecting away from the direction of forward rotation and towards the direction of backward rotation.

In embodiments described above, a pawl in the plurality of pawls has an angled surface facing a forward rotation direction and a concave curved surface facing away from the forward rotation direction.

In embodiments described above, a pawl in the plurality of pawls has an angled surface facing a forward rotation direction and a straight surface facing away from the forward rotation direction.

In embodiments described above, a pawl in the plurality of pawls has an angled surface facing a forward rotation direction and an angled surface facing away from the forward rotation direction.

In embodiments described above, the threads include a circumferential rim projecting from the proximal surface of the threads.

In embodiments described above, the proximal surface of the threads is curved in a concave manner.

In other embodiments, there may be at least one longitudinal channel defined within the propeller having an intake aperture defined in a proximal portion of the propeller and at least one output aperture defined along the shaft positioned distally from the intake aperture.

In other embodiments, there may be at least one longitudinal channel defined within the propeller having an intake aperture defined in a proximal portion of the propeller and a plurality of tubules branching from at least one longitudinal channel and ending in a plurality of output apertures defined along the shaft and positioned distal to the intake aperture.

In some embodiments discussed above, the cross-sectional area of the longitudinal channel varies along its longitudinal length.

In some embodiments discussed above, the cross-sectional areas of the tubules are variable in size to allow for a uniform deposition of material through the plurality of output apertures.

In some embodiments discussed above, an intake aperture is defined within a central bore located defined within the proximal end.

In the embodiments described above, the propeller comprises a distal portion and a proximal portion, wherein the distal portion includes a forward distal thread-form shape to assist in drilling through bony tissue during implant placement.

In yet other embodiments, there may be a pedicle anchoring device, comprising an anchoring having a propeller comprising a shaft having a first rotational axis; helical threads positioned about a longitudinal portion of the shaft forming a plurality of helical root valleys between crests of the helical threads, and the helical threads having distal surfaces and proximal surfaces; a plurality of pawls projecting from the distal surfaces to create a turbine-like thread-form shape; wherein the plurality of pawls are angled with respect to the distal surfaces to allow for forward rotation of the threads and to resist backward rotation of the threads; and an irregular lattice structure formed within the plurality of helical root valleys.

In yet other embodiments, there may be a method of placing a pedicle anchor into bony material, the method comprising: applying a torque to a proximal end of a propeller to rotate a propeller of the surgical implant about the propeller's longitudinal axis in a first rotational direction within a first bony structure to propel the implant in a first longitudinal direction; rotating a first plurality of rear-facing pawls in the first rotational direction to compress the bony material by the pawls while propelling the propeller forward; compressing bony material between a cupped proximal side of a first thread and the distal side of a second thread; and injecting a first internal labyrinth defined in the first propeller with bone growth material such that the bone-grown material flows from an opening defined in a proximal portion of the propeller into a plurality of channels and out of a plurality of openings defined along the shaft.

These and other features and advantages will be more clearly understood from the following detailed description taken in conjunction with the accompanying drawings. It is important to note that the drawings are not intended to represent the only aspect of the invention.

### BRIEF DESCRIPTION OF THE DRAWINGS

FIG. 1A. is a perspective view illustrating one embodiment of a pedicle anchoring system with a polyaxial head incorporating one or more aspects of the present invention.

FIG. 1B is an exploded perspective view of the pedicle anchoring system illustrated in FIG. 1A.

FIG. 1C is an exploded perspective view illustrating an anchor head and connection system of the system illustrated in FIG. 1A.

FIG. 1D is a detailed perspective section view illustrating an anchor head and connection system of the system illustrated in FIG. 1A.

FIG. 2 is a perspective view of a stabilization system illustrating the use of a rod and multiple pedicle anchors coupled to the rod.

FIG. 3A is a perspective view illustrating one embodiment of a pedicle anchor that incorporates one or more aspects of the present invention.

FIG. 3B is a proximal end view of the pedicle anchor illustrated in FIG. 3A.

FIG. 3C is a perspective side view of the pedicle anchor illustrated in FIG. 3A.

FIG. 3D is a perspective sectional view of the pedicle anchor illustrated in FIG. 3A cut along a center axis.

FIG. 3E is a detailed perspective view of a thread-form embodiment that may be employed by the pedicle anchor illustrated in FIG. 3A.

FIG. 3F is a perspective sectional view of the pedicle anchor illustrated in FIG. 3A cut through a plurality of thread forms.

FIG. 3G is a detailed perspective sectional view of the pedicle anchor illustrated in FIG. 3A cut through a single thread form.

FIG. 3H is a detailed perspective view of an alternative implant feature showing edge details of a thread.

FIG. 3I is a detailed perspective view of the proximal faces of a plurality of thread forms, which may be used by the pedicle anchor illustrated in FIG. 3A.

FIG. 3J is a perspective sectional view of the pedicle anchor illustrated in FIG. 3A, cut along a center axis, where the interior material is illustrated as transparent to show one embodiment of internal cannulas and passageways.

### DETAILED DESCRIPTION

For the purposes of promoting an understanding of the principles of the present inventions, reference will now be made to the embodiments or examples illustrated in the drawings, and specific language will be used to describe the same. It will nevertheless be understood that no limitation of the scope of the invention is thereby intended. Any alterations and further modifications in the described embodiments and any further applications of the principles of the inventions as described herein are contemplated as would normally occur to one skilled in the art to which the invention relates.

When directions, such as upper, lower, top, bottom, clockwise, or counter-clockwise, are discussed in this disclosure, such directions are meant to only supply reference directions for the illustrated figures and for orientation of components with respect to each other or to illustrate the figures. The directions should not be read to imply actual directions used in any resulting invention or actual use. Under no circumstances should such directions be read to limit or impart any meaning into the claims.

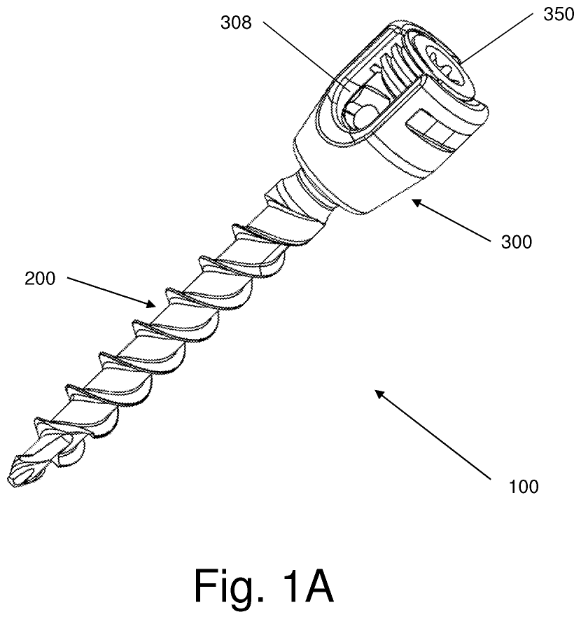

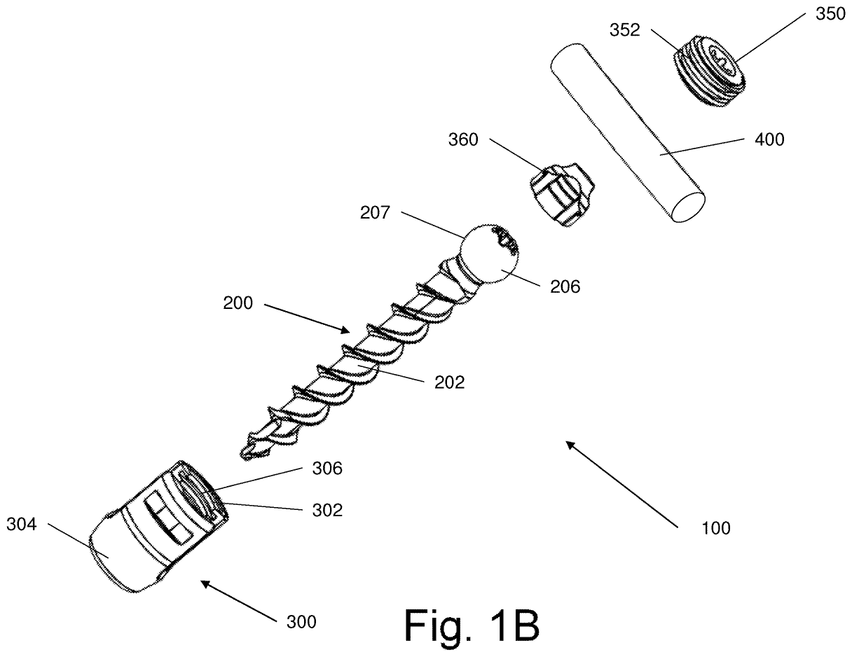

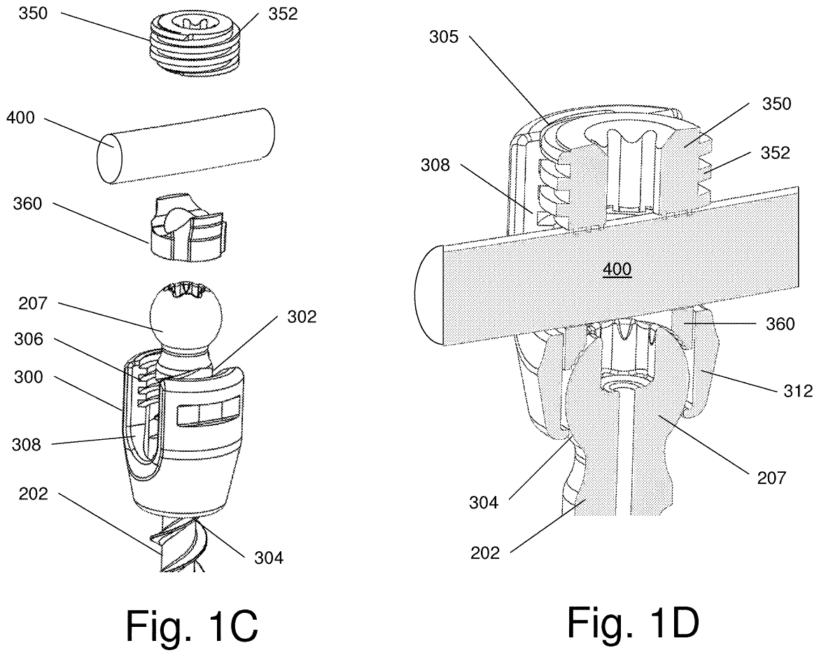

FIG. 1A illustrates one embodiment of an example pedicle anchor implant system 100, illustrating an anchor 200, a rod connector 300, and a retaining mechanism 350 (e.g., a set screw). FIG. 1B illustrates an exploded perspective view of the pedicle anchor implant system 100 showing the rod connector 300, the pedicle anchor 200, a swage-like fitting or “saddle” 360, a portion of a stabilizing rod 400, and the retaining mechanism, such as a set screw 350. FIG. 1C is a detailed exploded view of the proximal portion of the anchor 200 and rod connector, the saddle 360, a portion of the rod 400, and the set screw 350, illustrating the components of the connector system before final assembly. FIG. 1D is a section view of the connector system illustrating how the components work once assembled.

In the example illustrated in FIGS. 1A through 1D, the anchor 200 comprises an anchor shaft 202 and a proximal portion 206, which comprises a ball end 207. The rod connector 300 may be tubular in shape, defining a center bore 302. A lower or distal portion 304 of the center bore 302 is large enough to allow the passage of the anchor shaft 202 but small enough to capture the ball end 207, as best illustrated in FIG. 1D. The upper portion 306 of the center bore 302 is threaded and sized to mate with the external threads 352 formed on the external circular face of the set screw 350.

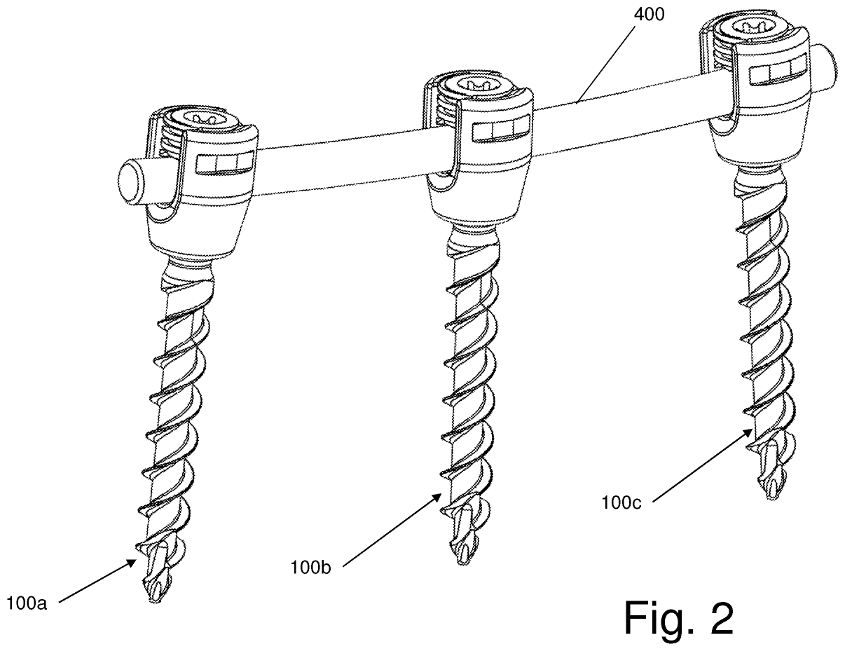

Two U-shaped slots 308 are defined within the walls of the rod connector 300. The U-shaped slots 308 are designed to allow the passage of the stabilizing rod 400, as best illustrated in FIGS. 1D and 2.

When assembled, the ball end 207 forms part of a ball and socket swivel joint with the lower portion 312 of the connector head 300, as best illustrated in FIG. 1D. The saddle 360 is positioned over the ball end 207. The saddle 360 is contoured where its surfaces contact both the ball end 207 and the rod 400. Thus, driving the set screw 350 against the internal threads of the upper portion of the center bore 305 causes a compressive force to be exerted on the rod 400, which causes compression to be exerted on the saddle 360. In turn, the saddle 360 exerts a compressive force on the ball end 207 and the socket formed by the lower portion 312 of the rod connector 300. Compression of the ball and socket, as well as the above-noted components, sets and secures their positional relationship to each other and locks the angular position of the ball end 207 and anchor shaft 202 with respect to rod 400 while also securing the rod in place.

As is known in the art, the rod 400 may be connected to one or more anchor systems 100a to 100c as illustrated in FIG. 2. For example, FIG. 2 illustrates an assembled two-level pedicle stabilization system comprising a single stabilization rod 400 and three pedicle screw systems 100a, 100b, and 100c.

Although polyaxial anchor systems have been discussed as example embodiments, any pedicle screw head system may be used with the inventive concepts of this disclosure and is within the scope of this invention. Such systems include fixed or monoaxial screw heads, dual-threaded heads, hybrid/adjustable heads, modular/universal heads, and magnetic heads, in addition to other pedicle screw head connection systems known or will be developed in the art. Similarly, any appropriately shaped proximal end or head may be used with the anchor. In other words, the inventive concepts disclosed herein are not limited to non-modular pedicle anchors with only ball-shaped heads.

In addition to the illustrated pedicle anchor systems 100a-100c, embodiments of the pedicle anchor system 100 and pedicle anchors 200 may be produced in a variety of other shapes, lengths, and sizes to accommodate the diverse anatomical and clinical conditions encountered in orthopedic surgery. Different patients present with varying bone densities, volumes, and geometries, as well as unique functional and aesthetic requirements, making a one-size-fits-all approach impractical. By offering a range of implant designs—such as varying diameters, lengths, and body contours—clinicians can select the most appropriate anchor to achieve optimal primary stability and preserve surrounding bone structures. Such variations are also within the scope of this invention.

### The Features of the Anchor

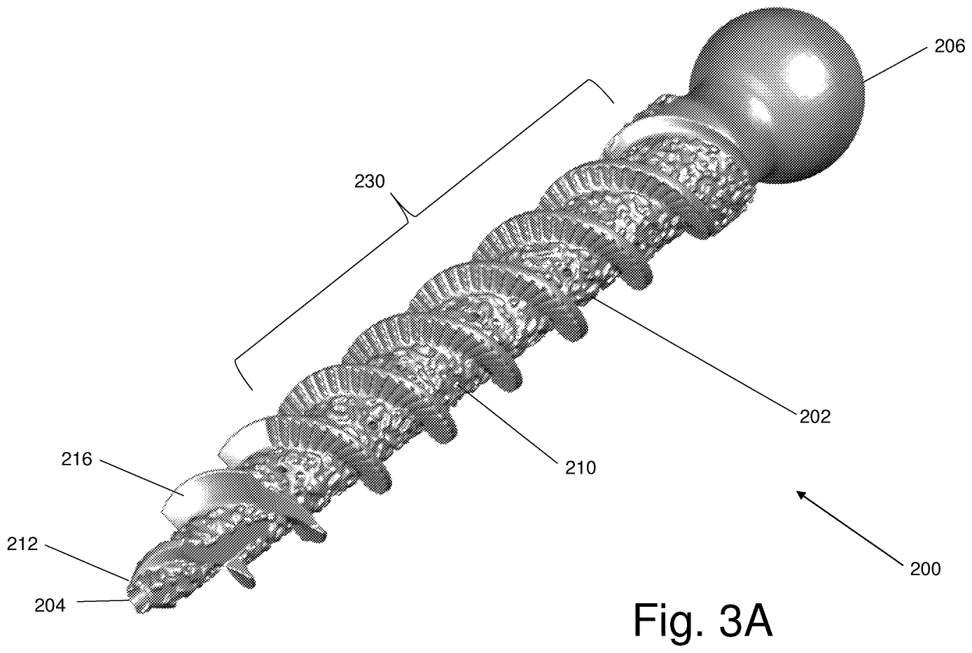

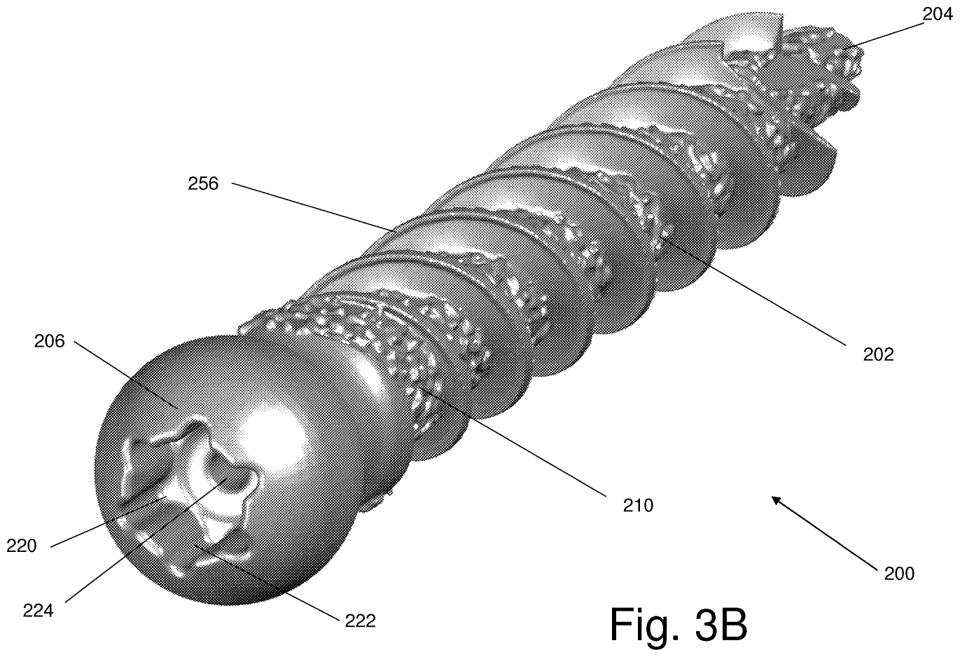

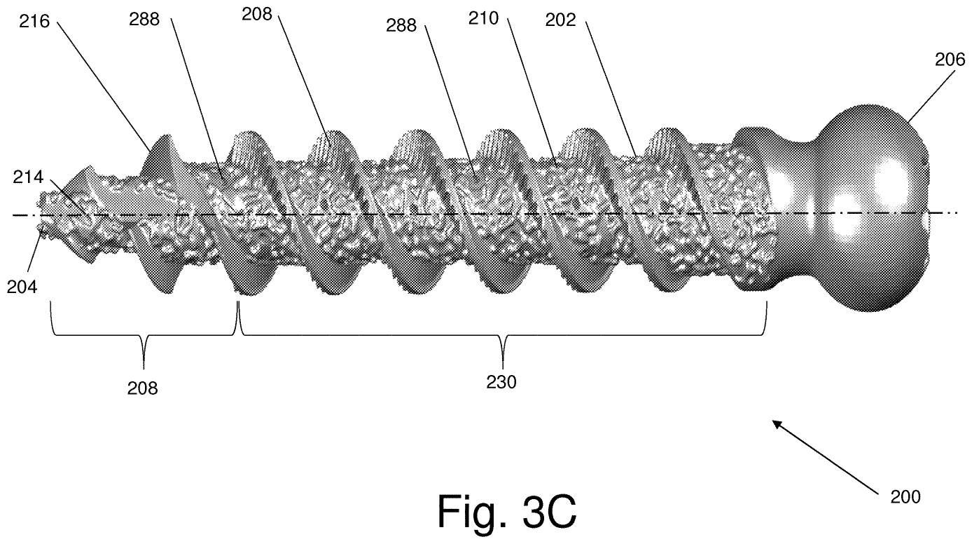

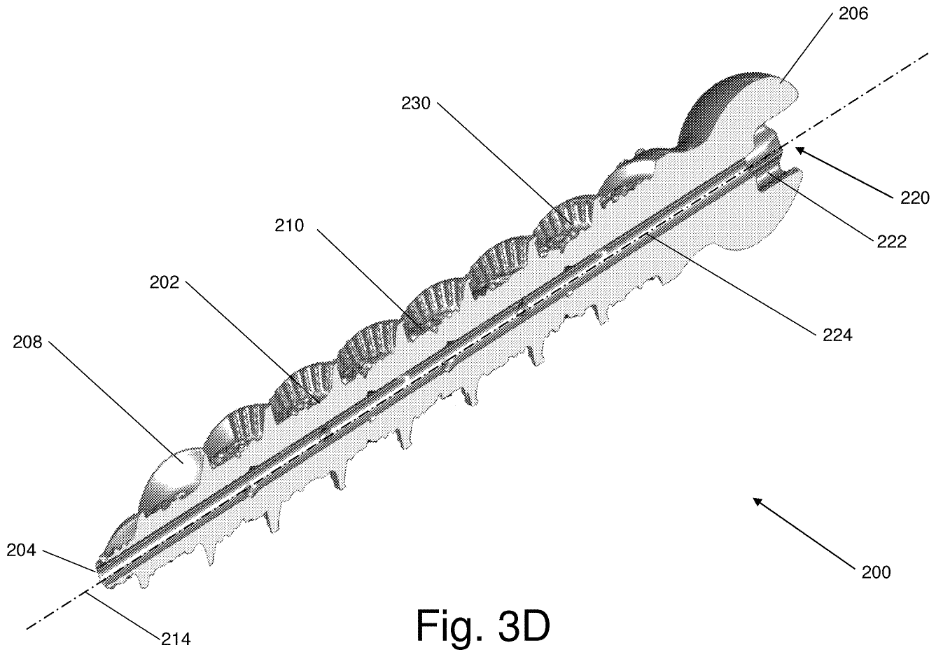

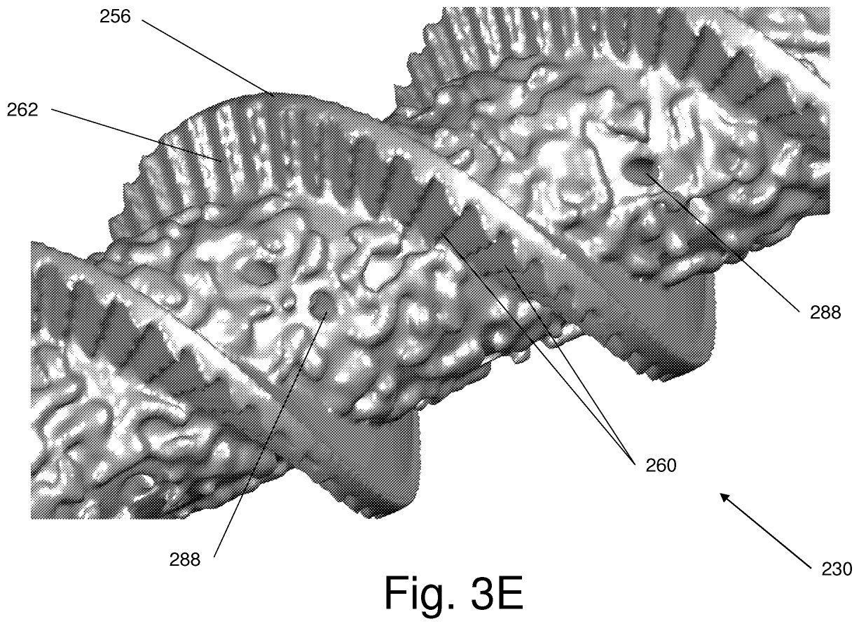

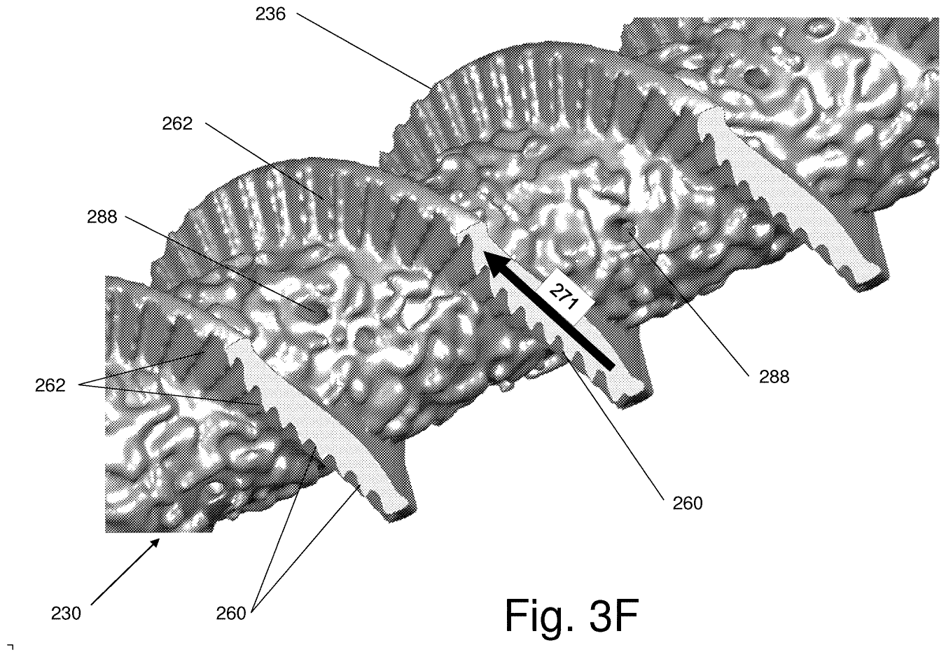

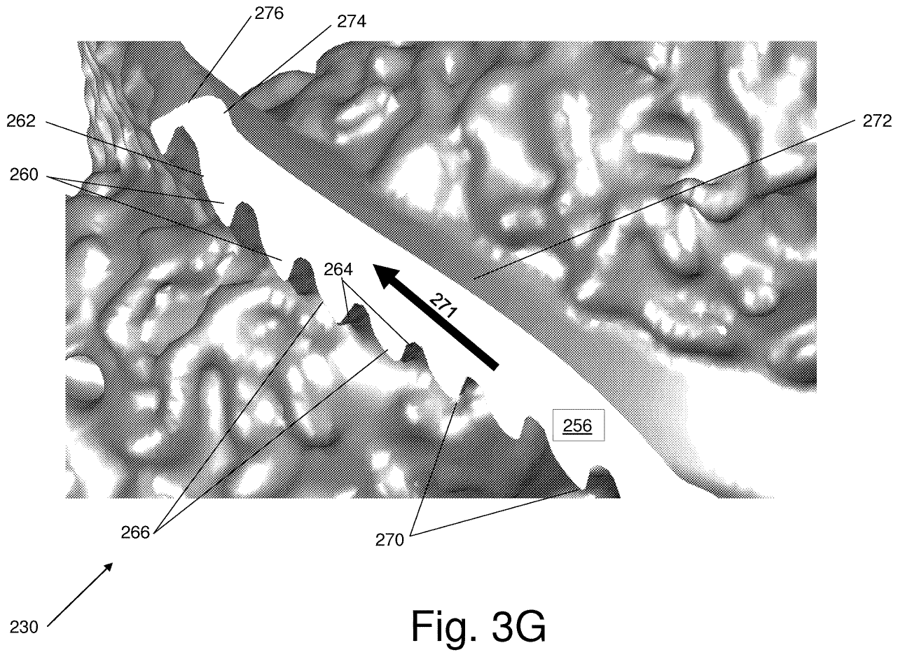

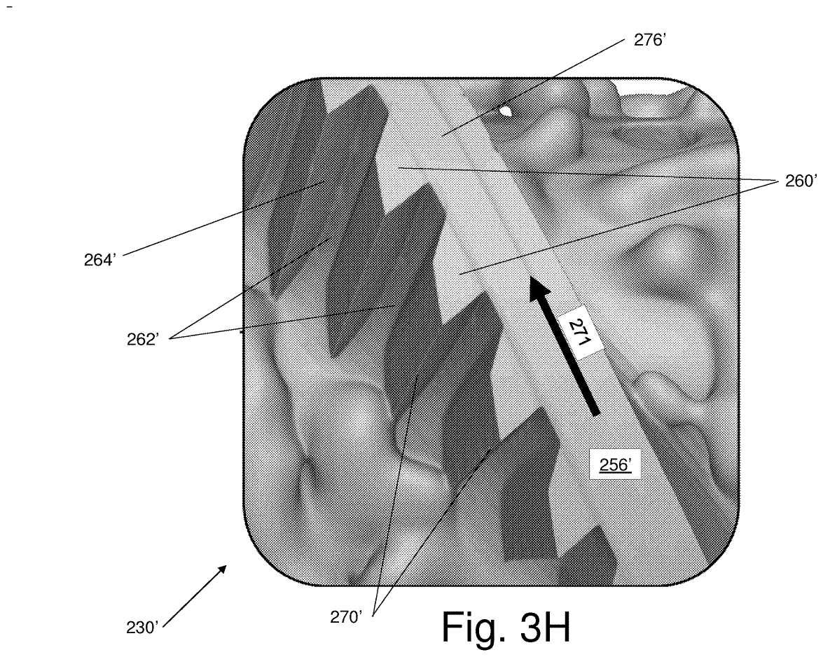

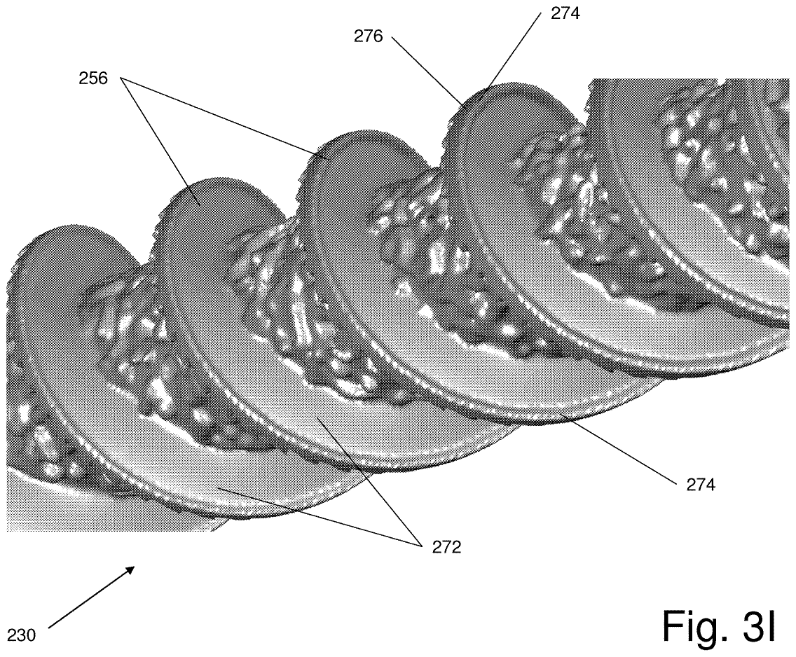

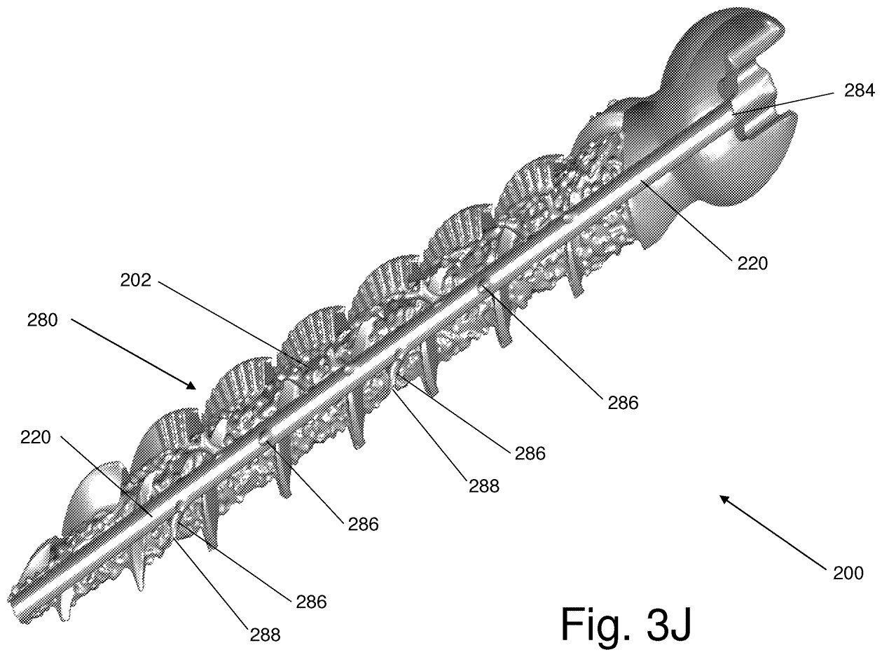

FIG. 3A is a perspective view of the anchor 200 as viewed from its distal end 204. FIG. 3B is an end view of the anchor 200 as viewed from a proximal end 206, and FIG. 3C is a side view of the anchor 200.

In certain embodiments, the anchor 200 comprises a center longitudinal shaft 202, as illustrated in FIGS. 3A and 3C.

As will be explained below in greater detail, in certain embodiments, one or more threads or thread flights 256 circumferentially encircle the shaft 202 in a helical manner, creating a series of helical root valleys and crests as illustrated in FIGS. 3B and 3C. In certain embodiments, there may be a porous lattice or fenestrated structure 210 that may be etched, milled, or three-dimensionally (3D) printed around the shaft 202 in the valleys or minor diameter (between the threads) of the anchor 200 to encourage bone growth. In embodiments, which include the internal labyrinth discussed below, the cannulas and channels of the labyrinth may end in apertures 288 defined in this lattice 210 (or slightly beyond the lattice) to further encourage bone growth in and around the shaft.

In certain embodiments, the distal end 204 of shaft 202 may have a pointed or penetrating surface 212 defined therein to penetrate and allow movement through bony tissue (not shown). In some embodiments, helical cutting threads 216 may also be positioned adjacent to the distal portion 204. In contrast, a proximal portion may act as a propeller region or propeller 230 and induce compression and drive the anchor forward when rotation is applied, as will be explained below in further detail.

FIG. 3D is a perspective section view of one embodiment of the anchor 200, showing a section cut approximately along the longitudinal or center axis 214 of the center shaft 202. In certain embodiments, a center bore or cannula 220 may be defined at the proximal end 206 of the center shaft 202, which runs longitudinally through the shaft 202 to the distal end 204 along a longitudinal axis 214.

In certain embodiments, the center bore 220 comprises two sections: a proximal torque engagement section or feature 222 and a smaller cannulated portion 224. The torque engagement section 222 is defined within the center bore 220 for engaging with a torque-inducing or driving device (not shown). For instance, in some embodiments, the torque engagement feature 222 may be a torx-shaped socket for engaging a similarly shaped torx driver of an insertion or driving instrument (not shown). In other embodiments, the engagement feature 222 may be hexagonal, octagonal, or trilobe, depending on the specific application and shape of the insertion instrument.

In the example embodiment illustrated in FIG. 3C, a first screw flight 208 may comprise a distal cutting surface and/or helical thread form 216 or helical flight designed to cut through bony material, and a “propeller” flight 230 designed to induce compression of the surrounding bone and to drive the anchor 200 through a bony substance when a torque is applied to the torque engaging feature 222.

The helical threads comprising the propeller region or propeller 230 have unique characteristics and features designed to increase propulsion force to propel the implant fixture 200 in a forward direction while creating compression in the surrounding bony tissue during and after placement. Thus, this helical flight creates greater propulsion and compression. For purposes of this disclosure, this “region of increased propulsion and compression” will be referred to as a propeller region or “propeller” 230.

In certain embodiments, there may be a single proximal thread form, flight, or propeller 230 defined throughout the entire length of the shaft 202. In other embodiments, there may be two, three, or even four flights (not shown) surrounding the center shaft 202, wherein each flight has different characteristics for either driving the implant through the bony material and/or for preventing backout (or a combination of both).

FIG. 3E is a detailed perspective view illustrating an example of aggressive helical thread form geometry in the propeller region 230 and is designed to increase the propulsion force to advance the anchor 200 and induce compression during placement. FIG. 3F is a partial section view of the helical thread form or propeller 230, where the section is cut through the threads to illustrate the unique shape of the thread forms in that section.

In certain embodiments, as illustrated in FIG. 3F, there may be an aggressive thread design characterized by relatively high crests 236 of the implant thread form showing a large radial difference between the minor diameter and the major diameter, which may be relatively large compared to the prior art. This difference helps to maximize the propulsive force as the propeller 230 propels the anchor 200 into the bony material and provides the compression of the bony material as the implant moves through the bone.

As discussed above, certain embodiments may include a cutting region 208 and/or penetrating point 212 immediately after the distal end 204. In certain embodiments, this threaded cutting region 208 and/or penetrating point 212 provides the requisite interaction with bony tissue (not shown) to cut and propel the anchor 200 forward until the threads of the propeller 230 can interact with the bony tissue - at which time, the propeller 230 may propel the implant forward.

### Thread Form Surface Features

FIG. 3E is a partial side perspective view of the propeller 230 of the anchor 200, illustrating certain distal facing or front side surface details. In contrast, FIG. 3F is a partial section view showing a cut through the threads of the propeller 230. FIG. 3G is a detailed sectional view of the thread forms where the longitudinal direction has been expanded for illustrative purposes to show additional details.

As illustrated in FIGS. 3E through 3G, in certain embodiments, there may be “pawls” or radial blades 260 projecting from the front or distal surfaces 262 of the threads 256 of the propeller 230 to create a turbine-like effect. As illustrated, in certain embodiments, the pawls 260 extend outwardly from the direction of the shaft in a somewhat radial manner, generally transverse to the circumferential direction of the thread of the propeller 230. In section, as can be seen in FIGS. 3F and 3G, the pawls 260 are generally triangular in cross-sectional shape, having a leading side or surface 266 pointed away from the direction of travel (arrow 271) and a back side or surface 264 meeting the leading side 266 to form a sharp edge 270. In certain embodiments, the sharp edge 270 may be at a predetermined angle (e.g., 20 degrees) relative to the distal surface 262 of the thread 256 and projects in a generally opposite direction from the forward rotation direction 271 (during placement of the anchor).

The geometry of the pawls 260 enables rotation in a single rotational direction (forward rotational travel 271) and prevents backout rotation, similar to a ratchet and pawl mechanism, because the sharp edges 270 will catch on the surrounding bony structure if the direction of travel is reversed. In certain embodiments, there may be subtle pitch changes of the pawls 260 along the threads, which will result in increased compression of the bony material.

Focusing now on FIG. 3G, there is a detailed section view cut through one of the threads 256. In certain embodiments, a backside surface 264 of the pawl 60 is curved. With the curved back surface 264, as the thread 256 is rotated, additional pressure is applied against the bony material, creating a turbine-like effect. In other words, as the anchor 200 is rotating into position, the pawls 260 may create constant pressure even at low rotation speeds. The pressure may increase slightly as the anchor 200 progresses so that the anchor 200 creates compression and retains itself as it is placed. With the curved back surface 264, as the thread 256 is rotated, additional pressure is applied against the bony material, creating a turbine-like effect. In other words, as the implant fixture 200 is rotating into position, the pawls 260 may create constant pressure even at low rotation speeds. The pressure may increase slightly as the implant fixture 200 progresses so that the implant fixture 200 creates compression and retains itself as it is placed. Furthermore, as rotation occurs, subtle pitch changes will occur along the threads, resulting in an increase in compression, and the “scooped” segments created by the back side of the pawls maximize surface area for bone ingrowth after placement.

FIG. 3H illustrates an alternative embodiment for the pawl and turbine system on an alternative thread and shows a perimeter edge 276′ of a single thread 256′ of an alternative propeller region 230'. As illustrated in FIG. 3H, the pawls or radial blades 260′ may be more defined than in the previous embodiment discussed above. Similar to pawls 260 discussed above, the pawls 260′ project from the front or distal surfaces 262′ of the thread 256′ to create a turbine-like effect. As illustrated, in certain embodiments, the pawls 260′ extend from the surface 262′ of the thread 256′ towards the distal end of the shaft 202 and generally transverse to the circumferential direction of the thread of the propeller 230. In section, as can be seen in FIG. 3H, the pawls 260′ form a sharp edge 270′, which faces away from the rotational direction of travel indicated by arrow 271. In certain embodiments, the sharp edge 270′ may be at a predetermined angle (e.g., 20 degrees) relative to the distal surface 262′ of the thread 256′.

The geometry of the pawls 260′ enables rotation in a single rotational direction 271 (forward rotational travel) and prevents backout, similar to the system described above, because the sharp edges 270′ will catch on the surrounding bony structure if the direction is reversed. In certain embodiments, there may be subtle pitch changes along the threads, which will result in increased compression of the bony material. In certain embodiments, the pawls may be evenly spaced throughout the faces of the threads. In other embodiments, the pawls may be spaced differently on the distal and proximal thread portions. For instance, the pawl spacing may increase along the thread flight from the distal threads to the proximal threads. In another embodiment, the pawl spacing may decrease along the thread flight from the distal to proximal threads. In addition, the pawl depth (from the face of the thread) and radial angle may be adjusted as necessary for the specific application. In yet other embodiments, the cross-section shape of the pawls may be a right triangle, an isosceles triangle, or another polygon. Furthermore, in some embodiments, the pawl projection path in the radial direction may be straight as illustrated or curved.

In certain embodiments, in cross-section view normal to the longitudinal direction, a backside surface 264′ of the pawl 260′ is curved or straight. The back side surface 264′ allows additional bone harvesting because of a cupping effect, which may also create subtle compression in the harvested bony material, while the thread 256′ of the propeller 230 is rotating to position the anchor 200. Furthermore, as the thread 256′ is rotated, additional pressure is applied against the bony material, creating a turbine-like effect as well as compression.

FIG. 3I is a partial side perspective view of a propeller 230 of the anchor 200, illustrating certain proximal facing or backside surface details of the threads 256. Turning now to FIG. 3I and FIG. 3G, the back side or proximal face 272 of the thread 256 is illustrated with a circumferential rim 274 projecting in a proximal direction around the perimeter 276 of the thread 256. Furthermore, in some embodiments, the proximal face 272 may be curved in a concaved manner, creating a cupping effect. During rotation occurring during implant placement, the rim 274 and the slight cupping may gather additional bony material between the threads, causing a pressure wave within the bony material, resulting in additional compression of the bony material. This “backside” geometry wedges the anchor into the bony material, resulting in increased compression of the bony material.

### Flowable Material Distribution System

As noted above, FIG. 3D is an isometric section view of the anchor 200. In contrast, FIG. 3J is an isometric section view of the anchor 200 where the interior solid material is shown in a transparent manner to illustrate certain interior channels, cannulas, structures, and details defined within the shaft 202.

As discussed above, the main central bore or cannula 220 runs along the anchor's longitudinal or center axis 214. The main cannula 220 may be used with guide wires for accurate placement of the anchor under fluoroscopy, as is known in the art. However, certain embodiments may include the main cannula 220 as part of an internal labyrinth structure 280 having a branched, tree-like appearance for distributing biologics and bone growth after implant placement. In certain embodiments, the structure or labyrinth 280 is formed from a plurality of tubules or channels 286 branching off the central cannula 220. The tubules 286 may be internally printed and flow from the center cannula 220 to apertures 288 defined on the surface of the shaft 202 and, in some embodiments, to the threads of the anchor 200 (See also FIGS. 2, 2C, 2F).

In certain embodiments, the initial diameter or size of the tubules 286 varies along the longitudinal axis to allow for relatively even distribution of the flowable biologic material. For instance, distal tubules 286 may have larger diameters where the pressure in the center cannula 220 is less to allow the biologics to flow evenly throughout the entire structure. In certain embodiments, the tubules branch or split into smaller tubules and exit at the minor diameter of the anchor. In yet other embodiments, the tubules 286 extend into the thread forms (not shown). The tubule size and distribution may be customized for optimal graft flow—depending on the application and the flowability of the injected material.

In certain embodiments, pressurized biological material, such as treated cadaverous bone material, may be injected into the center cannula 220 via an opening 284 in the center of the torque engagement feature 222 from a syringe or another pressure-inducing device. The material will then flow down the center cannula and into the various branches of the labyrinth and, in the illustrative embodiment, out into the shaft. In certain situations, certain medicines, such as analgesics or antibiotics, may also be injected into the center cannula to relieve post-operative discomfort and/or to prevent infection.

### Manufacturing Methods and Materials

In certain embodiments, the implants (such as anchor 200) may be manufactured utilizing three-dimensional (3D) printing, where the implant is printed as a relatively complete assembly incorporating the external thread form and internal structures in the anchor 200. If necessary, such embodiments may then be finalized with standard machining methods to clean up or add various surfaces and features.

In certain embodiments, the implants and anchors discussed above may be fabricated from any number of biocompatible implantable materials, including but not limited to Titanium Alloys (Ti 6Al4V ELI, for example), commercially pure titanium, Chromium Cobalt (Cr—Co), stainless steels, and ceramics. In other embodiments, the implants and anchors may also be manufactured from polymer, including Carbon Fiber Reinforced Polymer (“CFRP”) with a high carbon mass percentage. In some embodiments, the implants (or portions of the implants) may be coated with a bone-conducting surface treatment to increase the potential of bone on-, through-, or in-growth.

### OPERATION

The manner of using certain aspects of the present invention will now be described. In certain embodiments, the patient is positioned prone on a radiolucent table, and a sterile midline incision is made over the targeted spinal segments. The surgeon then dissects the paraspinal muscles subperiosteally to expose the posterior elements of the vertebrae, such as the lamina, facet joints, and pedicles. Using fluoroscopy or navigation guidance, the pedicle entry points are carefully identified, and a guide wire is inserted through a pilot hole into the pedicle to establish a pathway. In certain embodiments, a burr or drill may then be used along the guide wire to create a pilot opening.

In certain embodiments, a blunt pedicle probe may be advanced along the guide wire to confirm proper canal cannulation. The anchors 200 may then be inserted over the guide wire into the prepared pedicles, and advanced into the prepared bone site using a special implant driver or wrench (not shown) which has a torque feature designed to mate with and apply torque to the torque engagement feature 224 of the central bore 220 of the anchor 200 as described above.

Applying controlled torque with the driver rotates the propeller 230 of the anchor 200. As the anchor 200 is rotated, a plurality of pawls 260 projecting from the distal surface of helical threads act as a turbine and propel the anchor 200 forward while causing compression on the surrounding bone. As described above, a rotation of the rim 274 and concave curved surface on the proximal side of the threads tends to harvest some of the surrounding bony tissue between the threads, causing compression between the implant and the bony structure surrounding the implant.

After the anchors are positioned, the guide wires are removed, and bone growth material, such as demineralized bone matrix or other osteoinductive substances, is applied into the central bore 220 and distributed throughout the anchor as described above.

Subsequently, the surgeon contours the rod 400 to fit the spine's anatomy, then places it into the screw heads. The set screws 350 are then tightened to secure the construct. After ensuring stability and correct positioning, the wound is irrigated, and muscles and skin layers may be closed in sequence. Postoperative imaging confirms hardware placement, and the patient is monitored for recovery.

### Advantages of Certain Embodiments

Conventional pedicle anchors have predominantly focused on optimizing mechanical fixation through design modifications; however, biological factors such as bone quality and the interface's healing potential significantly influence their stability. Insufficient osseointegration can lead to loosening, micro-movement, and eventual failure of the implant, particularly in cases involving osteoporotic or compromised bone. The disclosed propeller is designed to penetrate both osteopenia bone and sclerotic bone without the need for the use of percussive force.

The compressive features of the thread form and propeller discussed above may cause subtle compression during the application of a smooth force used to insert the propeller. In certain situations, subtle compression may be preferred because the bony structure may not be able to withstand aggressive compression. Not only does the insertion of the propeller initially cause compression of the bony material immediately surrounding the propeller, compression continues after placement - resulting in more rapid healing when compared to conventional pedicle implants.

In sum, by applying controlled compression, the disclosed system can increase circumferential bone contact, promote dense bone-screw apposition, and stimulate early and robust osseointegration. This biomechanical strategy not only stabilizes the implant immediately post-surgery but also fosters favorable biological responses conducive to long-term integration.

It is also important to realize that although the present invention is disclosed in terms of a pedicle anchor system, the unique screw threads, pawls, and compression systems disclosed have applications anywhere throughout the body for a variety of applications and procedures. Thus, the scope of this invention is not limited to pedicle anchors or pedicle implant systems.

It should be emphasized that while the present invention is primarily described in the context of a pedicle anchor or screw system, its innovative features and components have far-reaching potential beyond pedicle screw applications. The unique screw threads, pawls, and compression systems disclosed in this invention possess versatile characteristics that make them suitable for a wide array of medical and surgical applications throughout the human body. These components can be adapted and utilized in various orthopedic procedures, such as bone fracture fixation, and joint replacement surgeries. The novel design elements of these components offer enhanced stability, improved load distribution, and optimized tissue integration, which are beneficial in numerous anatomical locations and diverse clinical scenarios.

The foregoing description of the embodiments of the invention has been presented for the purposes of illustration and description. It is not intended to be exhaustive or to limit the invention to the precise form disclosed. Many combinations, modifications, and variations are possible in light of the above teaching. For instance, in certain embodiments, each of the above-described components and features may be individually or sequentially combined with other components or features and still be within the scope of the present invention. Undescribed embodiments that have interchanged components are still within the scope of the present invention. It is intended that the scope of the invention be limited not by this detailed description but rather by the claims.

For instance, in some embodiments, there is a sophisticated pedicle anchor device designed to enhance stability, ease of placement, and integration with surrounding bone tissue, significantly advancing traditional implant methodologies.

Embodiments of the pedicle anchor device comprise several unique components including an advanced anchor. Embodiments of the anchor are engineered with a shaft that features a rotational axis equipped with helical threads that include a series of interspersed root valleys having an irregular and porous lattice structure, designed to promote osseointegration by encouraging bone in-growth and providing additional fixation support.

The threads have turbine-like pawls projecting from their distal surfaces. These pawls are angled to enable effective forward rotational movement while resisting unintentional backward rotation. This is designed so that once the implant is positioned, it remains secure, reducing the risk of displacement. The pawls may feature various configurations, including angled, concave, or sharp edges, tailored to compress the bone and enhance grip during the implantation process, and thereafter.

Some embodiments include a channel system for bone-growth material delivery. These channels are longitudinally defined within the anchor, with design variations allowing for intake apertures and multiple output points along the shaft. This system enables the uniform delivery of bone growth materials directly into the surrounding bone structure, promoting enhanced integration and stability over time.

During placement, torque is applied to rotate the propeller within the bone. This motion, aided by the rear-facing pawls, compresses the bone material, enhancing initial stability. Furthermore, the device's design allows for the injection of bone growth material, channeled through an internal labyrinth to reach specific areas, maximizing biological integration and facilitating faster recovery.

In conclusion, this invention provides a comprehensive solution for bone anchor systems, utilizing advanced mechanical and material distribution designs to improve implant success rates, accelerate the healing process, and ensure a lasting bond with the bony structure.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

What is claimed is:

1. A bone implant device, comprising:
a connector,
a retaining mechanism; and
an anchor, including:
a shaft having a rotational axis;
distal portion including a thread-form shape to assist in drilling through bony tissue during implant placement;
a proximal portion forming a propeller, the propeller including,
helical threads positioned about a longitudinal portion of the shaft forming a plurality of helical root valleys between crests of the helical threads, and the helical threads having distal surfaces and proximal surfaces;
a plurality of pawls projecting from the distal surfaces to create a turbine-like thread-form shape;
wherein the plurality of pawls are angled with respect to the distal surfaces to allow for forward rotation of the threads and to resist backward rotation of the threads;
a circumferential rim projecting from the proximal surface of the helical threads and wherein the proximal surfaces of the helical threads are curved in a concave manner, and
an irregular porous lattice structure formed within the plurality of helical root valleys.

2. The bone implant device of claim 1, wherein at least one pawl in the plurality of pawls has an angled surface facing a forward rotation direction and a concave curved surface facing away from the forward rotation direction.

3. The bone implant device of claim 1, wherein at least one pawl in the plurality of pawls has a sharp edge projecting away from a direction of forward rotation and towards a direction of backward rotation.

4. The bone implant device of claim 1, wherein at least one pawl in the plurality of pawls has an angled surface facing a forward rotation direction and a concave curved surface facing away from the forward rotation direction.

5. The bone implant device of claim 1, wherein at least one pawl in the plurality of pawls has an angled surface facing a forward rotation direction and a straight surface facing away from the forward rotation direction.

6. The bone implant device of claim 1, wherein at least one pawl in the plurality of pawls has an angled surface facing a forward rotation direction and an angled surface facing away from the forward rotation direction.

7. The bone implant device of claim 1, further including at least one longitudinal channel defined within the anchor having an intake aperture defined in a proximal portion of the anchor and at least one output aperture defined along the shaft positioned distally from the intake aperture.

8. The bone implant device of claim 1, further including at least one longitudinal channel defined within the anchor having an intake aperture defined in a proximal portion of the anchor and a plurality of tubules branching from the at least one longitudinal channel and ending in a plurality of output apertures defined along the shaft and positioned distal to the intake aperture.

9. The bone implant device of claim 8, wherein a cross-sectional area of the longitudinal channel varies along its longitudinal length.

10. The bone implant device of claim 8, wherein cross-section areas of the tubules are variable in size to allow for a uniform deposition of material through the plurality of output apertures.

11. The bone implant device of claim 8, wherein the intake aperture is a central bore defined along the anchor's rotational axis.

12. A bone anchor, comprising:
a distal portion having a bone-cutting portion, and
a proximal propeller portion comprising:
a shaft having a rotational axis;
helical threads positioned about a longitudinal portion of the shaft forming a plurality of helical root valleys between crests of the helical threads, and the helical threads having distal surfaces and proximal surfaces;
a plurality of pawls projecting from the distal surfaces to create a turbine-like thread-form shape;
wherein the plurality of pawls are angled with respect to the distal surfaces to allow for forward rotation of the threads and to resist backward rotation of the threads;
a circumferential rim projecting from the proximal surface of the helical threads and wherein the proximal surfaces of the helical threads are curved in a concave manner, and
an irregular lattice structure formed within the plurality of helical root valleys.

13. A method of placing an anchor in bony material, the method comprising:
applying a torque to a proximal end of the anchor to rotate a propeller about the anchor's longitudinal axis in a rotational direction within a bony structure to propel the anchor in a longitudinal direction and resulting in a rotation of a plurality of pawls to compress the bony material while propelling the propeller forward;
compressing bony material between a cupped proximal side of a first thread of the propeller and a distal side of a second thread of the propeller; and
injecting a internal labyrinth defined in the propeller with bone-growth material such that the bone-growth material flows from an opening defined in a proximal portion of the anchor into a plurality of channels and out of a plurality of openings defined along a shaft of the anchor.

14. The method of claim 13, wherein the injecting the internal labyrinth is injecting the bone-growth material into a central cannula such that the bone-growth material flows from an intake opening defined in a proximal portion of the anchor into a plurality of channels and out of a plurality of openings defined along a shaft of the anchor.
